(19) United States
(12) Patent Application Publication
(10) Pub. No.: US 2019/0200889 A1
(43) Pub. Date: Jul. 4, 2019

(54) Electrical Activity Monitoring Systems and Methods for Determining Physiological Characteristics with Same

(71) Applicant: Medical Design Solutions, Inc., Milpitas, CA (US)

(72) Inventor: Robert T. Stone, Sunnyvale, CA (US)

(21) Appl. No.: 16/295,528

(22) Filed: Mar. 7, 2019

Related U.S. Application Data
(63) Continuation-in-part of application No. 15/491,775, filed on Apr. 19, 2017, now abandoned.

(51) Int. Cl. A61B 5/0408; A61B 5/00; A61B 5/0452

(52) U.S. Cl. CPC A61B 5/04085; A61B 5/0024; A61B 5/7203; A61B 5/0006; A61B 5/6804; A61B 5/0452; A61B 2562/182; A61B 2562/0214; A61B 2562/0215; A61B 5/6823

(57) ABSTRACT

An electrical activity monitoring system that includes an sensor sub-system having first and second capacitive electrodes that are configured to electrically couple to a subject's skin and detect electrical signals of a subject, a conductive layer is configured to couple to an anatomical region of the subject and generate an anatomical reference signal, a control module that is programmed to control the sensor sub-system and determine physiological characteristics as a function of signals transmitted from the sensor sub-system.

### CROSS-REFERENCE TO RELATED APPLICATIONS

This application is a continuation-in-part application of U.S. application Ser. No. 15/491,775, filed on Apr. 19, 2017, which claims priority to U.S. Provisional Application No. 62/325,290, filed on Apr. 20, 2016.

### FIELD OF THE INVENTION

The present invention relates to systems and methods for monitoring physiological characteristics of a subject. More particularly, the present invention relates to systems for monitoring electrical activity of a subject and methods for determining physiological characteristics of the subject as a function of the subject's electrical activity.

### BACKGROUND OF THE INVENTION

As is well known in the art, electrical activity of a subject provides an effective means for determining one or more physiological characteristics associated with the subject; particularly, physiological characteristics associated with the subject's heart. By monitoring the electrical activity of a subject, any abnormalities in a seminal physiological function or characteristic, e.g., heart rate and rhythm of heartbeats, can be determined and conveyed to a physician for prompt preventative action(s).

Various systems have thus been developed to monitor the electrical activity of a subject. The conventional systems are typically designed and configured to record the electrical activity of a subject's heart over a period of time and generate visual patterns or tracings representing the electrical activity of the subject's heart, known as electrocardiography (ECG). The patterns representing the electrical activity of the subject's heart are generally referred to as ECG patterns or signals.

As is well known in the art, during each heartbeat, a healthy heart has an orderly progression of depolarization that starts with pacemaker cells in the sinoatrial node, spreads throughout the atrium, passes through the atrioventricular node down into the bundle of His, i.e. a collection of heart muscle cells specialized for electrical conduction, and into the Purkinje fibers, spreading down and to the left throughout the ventricles. This orderly pattern of depolarization gives rise to a characteristic ECG pattern.

Referring now to FIG. 1, there is shown a characteristic ECG pattern (or signal) representing a heart in normal sinus rhythm. As illustrated in FIG. 1, there are three main components of an ECG pattern: the P wave, which represents the depolarization of the atria; the QRS complex, which represents the depolarization of the ventricles; and the T wave, which represents the repolarization of the ventricles.

The ECG pattern can also be further broken down as follows: P represents the atrial systole contraction pulse; Q represents a downward deflection immediately preceding the ventricular contraction; R represents the peak of the ventricular contraction; S represents the downward deflection immediately after the ventricular contraction; and T represents the recovery of the ventricles.

An ECG pattern thus conveys a large amount of information about the structure of the heart and the function of its electrical conduction system, including the rate and rhythm of heartbeats, the size and position of the heart chambers, and the presence of any damage to the heart's muscle cells or conduction system

As indicated above, various systems have thus been developed to record the electrical activity of a subject's heart over a period of time and generate visual patterns or tracings representing the electrical activity (referred to hereinafter as “ECG based systems”).

Illustrative are the ECG based systems disclosed in U.S. application Ser. No. 15/062,865, which employ a plurality of core electrodes that are adapted to measure the heart's electrical potential at various attachment sites and generate signals representing same, and a reference electrode that is typically positioned on the right leg of a monitored subject.

The reference electrode provides a low-impedance connection to a subject's body and generates and transmits a reference potential signal to a system control module, which is employed as a comparative parameter to adjust, if necessary, the electrical potential signals transmitted by the core electrodes.

There are numerous disadvantages and drawbacks associated with the use of a conventional reference electrode, including the sub-optimal placement of the reference electrode on the right leg. Indeed, as is well established, the reference electrode will often produce reference potentials that fail to account for the electrical potential of each individual anatomical structure, e.g., organs and muscles, disposed between the reference electrode and the core electrodes. As a result, the function of the associated ECG based systems can, and often will be, negatively impacted, resulting in lower signal quality due to baseline wandering and low-frequency artifacts.

A further drawback of conventional reference electrodes is that the reference electrodes will also often increase interference in and of signal amplifiers of ECG based systems.

Various ECG based systems have thus been developed to improve signal quality and accuracy of physiological characteristics determined thereby. Illustrative are the ECG systems disclosed in U.S. application Ser. Nos. 14/750,083 and 14/911,122.

U.S. application Ser. No. 14/750,083 discloses a compact ECG based system incorporated into a wristband that comprises two (2) capacitive electrodes and a third electrode that is employed as an “impedance path to ground” reference electrode to reduce the common-mode electrical potential noise in the signals received by the capacitive electrodes.

U.S. application Ser. No. 14/911,122 discloses a multi-layer capacitive textile electrode patch system comprising two electrically conductive layers of a textile material, a plurality of insulating layers, a guard layer and a reference potential layer in communication with a ground reference.

There are also several major drawbacks and disadvantages associated with the above noted ECG based systems. Among the disadvantages are the small size of the reference electrodes compared to conventional reference electrodes and the proximity of the reference electrode to the capacitive electrodes, which limits the size of the reference site used by the reference electrode to generate a reference potential.

By virtue of the above noted drawbacks, the function of the ECG based systems are similarly negatively impacted, resulting in poor signal quality due to baseline wandering, low-frequency artifacts and signal interference due to the proximity of the reference electrode to the capacitive electrode(s).

It would thus be desirable to provide improved ECG based systems that detect signals representing electrical activity of a subject with enhanced accuracy and record signals representing same that exhibit enhanced signal quality.

It is therefore an object of the present invention to provide improved ECG based systems that detect signals representing electrical activity of a subject with enhanced accuracy and record signals representing same that exhibit enhanced signal quality.

It is another object of the present invention to provide improved physiological monitoring systems that substantially reduce or abate external and internal signal interference.

It is another object of the invention to provide improved methods for accurately determining one or more physiological characteristics associated with a subject as a function of detected electrical activity.

### SUMMARY OF THE INVENTION

The present invention is directed to systems for monitoring electrical activity of a subject (referred to herein as “electrical activity monitoring systems”) and methods for determining physiological characteristics of the subject as a function of the subject's electrical activity.

In a preferred embodiment of the invention, the electrical activity monitoring systems comprise a sensor sub-system, signal transmission conductors and a control module.

In some embodiments, the sensor sub-system comprises multiple capacitive electrodes and a conductive layer.

In a preferred embodiment, the capacitive electrodes are designed and configured to electrically couple to a subject's skin and detect the electrical potential between the capacitive electrodes and the subject's skin, i.e. electrical activity, and generate capacitive potential signals representing same.

In a preferred embodiment, the capacitive electrodes are further designed and configured to detect capacitive potential noise proximate the electrodes, and generate capacitive potential noise signals representing same.

In a preferred embodiment of the invention, the conductive layer is adapted and configured to couple to an anatomical region of a subject, i.e. a capacitive reference site, and generate at least one pre-processed anatomical reference signal.

In a preferred embodiment, the conductive layer provides an electromagnetic shield for the capacitive electrodes.

In a preferred embodiment, the electrical activity monitoring systems further comprise a control module, which preferably includes a processing system that is programmed and configured to control the electrical activity monitoring systems and the function thereof, and the transmission and receipt of signals therefrom.

In preferred embodiment of the invention, the processing system is further programmed and configured to receive the capacitance potential signals, capacitance potential noise signals, and anatomical reference signals, and determine physiological information associated with the monitored subject as a function of the signals, including at least one physiological parameter or characteristic, e.g., heart rate.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of a characteristic electrocardiograph (ECG) pattern representing a heart in normal sinus rhythm;

FIG. 2 is a schematic illustration of one embodiment of an electrical activity monitoring system, in accordance with the invention;

FIG. 3A is a front plan, sectional view of one embodiment of a capacitive electrode, in accordance with the invention;

FIG. 3B is a perspective view of another embodiment of a capacitive electrode, in accordance with the invention;

FIG. 4 is a perspective view of one embodiment of a wearable electrical activity monitoring system employing the sensor sub-system shown in FIG. 2, in accordance with the invention; and

FIG. 5 is a perspective view of another embodiment of a wearable electrical activity monitoring system employing the sensor sub-system shown in FIG. 2, in accordance with the invention;

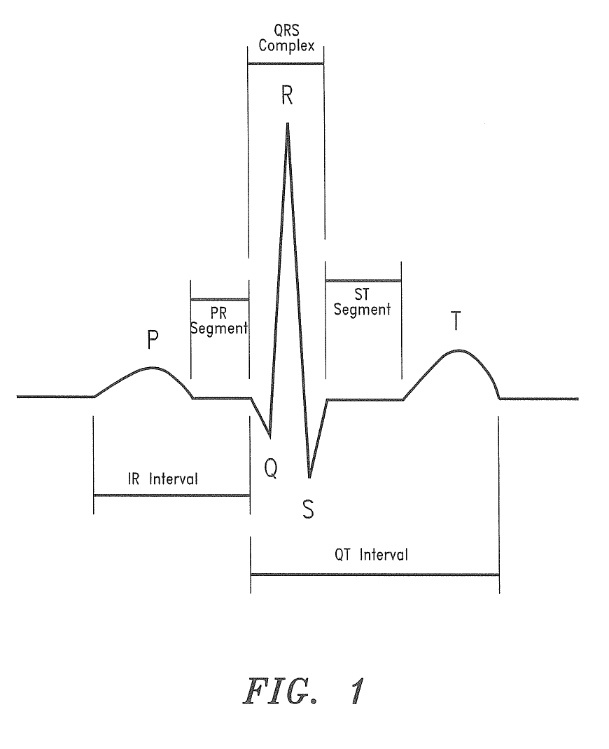

### DETAILED DESCRIPTION OF THE PREFERRED EMBODIMENT

Before describing the present invention in detail, it is to be understood that this invention is not limited to particularly exemplified apparatus, systems or methods as such may, of course, vary. Thus, although a number of apparatus, systems and methods similar or equivalent to those described herein can be used in the practice of the present invention, the preferred apparatus, systems, and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a sensor signal” includes two or more such signals and the like.

### Definitions

The term “physiological characteristic”, as used herein, means and includes any physical parameter associated with a warm-blooded mammal, including, without limitation, electrical activity of the heart, electrical activity of other muscles, electrical activity of the brain, pulse rate, blood pressure, blood oxygen saturation level, skin temperature, and core temperature.

The term “subject”, as used herein, means and includes warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to systems for monitoring electrical activity of a subject and methods for determining physiological characteristics of the subject as a function of the subject's electrical activity.

Although the electrical activity monitoring systems and associated methods of the invention are described in connection with monitoring and recording cardiac electrical activity, i.e. electrical activity of a heart, and methods for determining cardiac characteristics (and/or functions) based thereon, it is understood that the electrical activity monitoring systems and associated methods of the invention can also be readily employed to monitor and record electrical activity of other organs and structures, and determine physiological characteristics and/or functions based thereon.

It is also understood that, although the electrical activity monitoring systems and associated methods of the invention are described herein in connection with monitoring electrical activity of a human, the invention is not limited to such use. Indeed, the electrical activity monitoring systems and associated methods of the invention can also be readily employed to monitor electrical activity of other mammals.

It is further understood that, although the electrical activity monitoring systems and associated methods of the invention are described herein in connection with a wearable garment or band, the invention is not limited to such apparatus and/or applications. The electrical activity monitoring systems and associated methods of the invention can also be employed in other apparatus and systems that are configured to directly communicate with a subject's body.

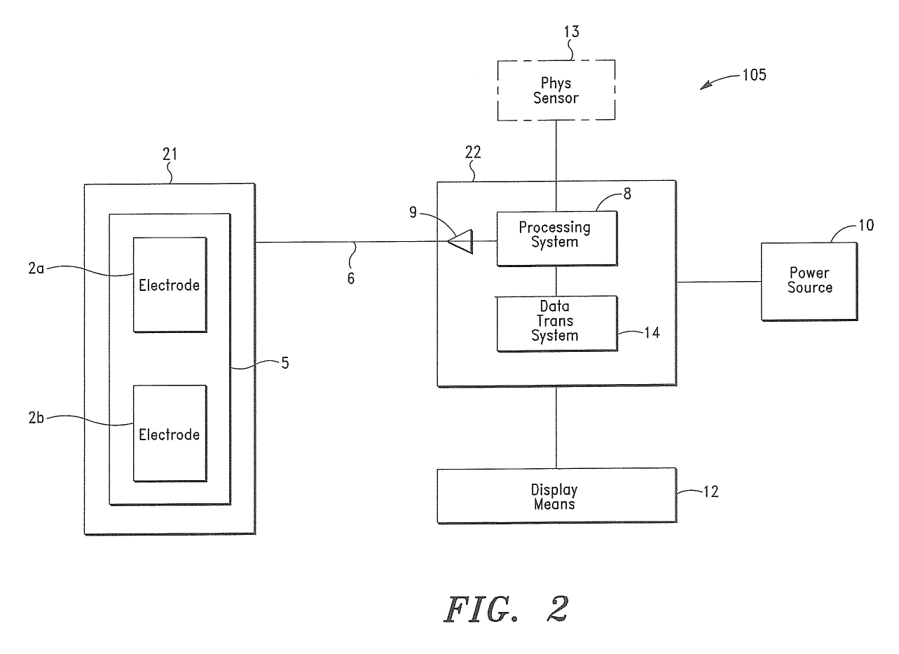

Referring now to FIG. 2, there is shown one embodiment of an electrical activity monitoring system of the invention. As illustrated in FIG. 2, the electrical activity monitoring system (denoted “105”) includes a sensor sub-system (or assembly) 21, signal transmission conductors 6, a control module 22 and a power source 10.

According to the invention, the power source 10 can comprise any device or system configured to provide (or generate) electrical energy, such as a battery.

As further illustrated in FIG. 2, the sensor sub-system 21 comprises first and second electrodes 2a, 2b and conductive layer 5.

As indicated above, in a preferred embodiment, the first and second electrodes 2a, 2b comprise capacitive electrodes that are designed and configured to electrically couple to a subject's skin and detect electrical potentials between the capacitive electrodes and the subject's skin, i.e. electrical activity, and generate capacitance potential signals representing same.

According to the invention, the first and second electrodes 2a, 2b can be electrically coupled to any anatomical region of a subject.

As also indicated above, in a preferred embodiment, the capacitive electrodes are further designed and configured to detect capacitive potential noise proximate the electrodes, and generate capacitive potential noise signals representing same.

According to the invention, the sensor sub-system 21 can comprise any number of capacitive electrodes. Thus, in some embodiments of the invention, the sensor sub-system 21 comprises a single capacitive electrode.

As indicated above, in a preferred embodiment, the sensor sub-system 21 comprises two (2) capacitive electrodes, i.e. capacitive electrodes 2a and 2b, as shown in FIG. 2.

In some embodiments, the first and second capacitive electrodes 2a, 2b comprise an area in the range of 0.5-20 cm², more preferably, the first and second capacitive electrodes 2a, 2b comprise an area in the range of 1.0-10 cm².

In a preferred embodiment, the first and second capacitive electrodes 2a, 2b are configured to accurately detect capacitance potential signals at electrode-to-skin distances in the range of 0.05-10 mm, more preferably, detect capacitance potential signals at electrode-to-skin distances in the range of 0.1-1.0 mm.

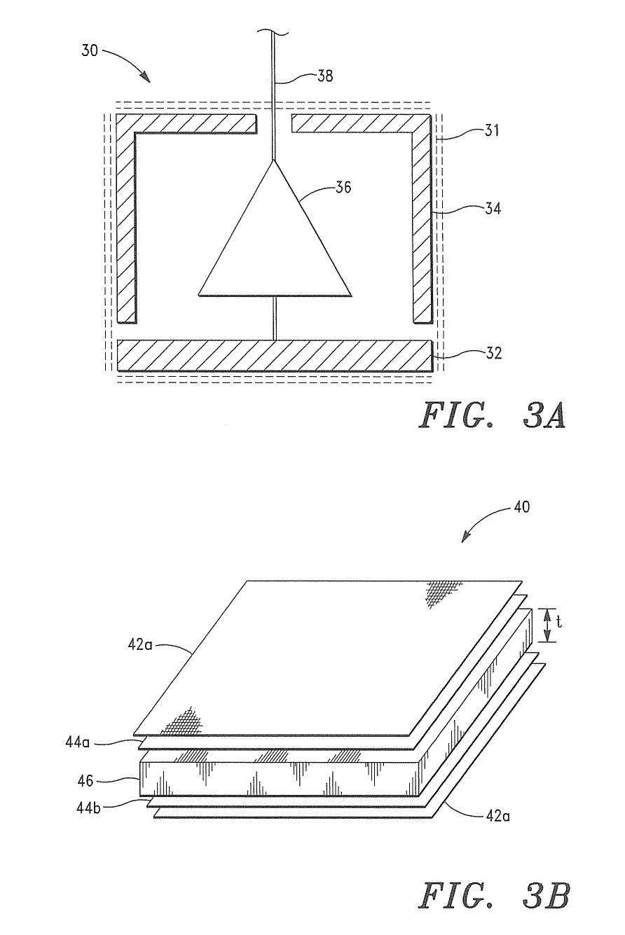

Referring now to FIGS. 3A and 3B, two (2) embodiments of capacitive electrodes of the invention will be described in detail.

Referring first to FIG. 3A, there is shown one embodiment of a capacitive electrode comprising a self-contained (SC) capacitive electrode 30. As illustrated in FIG. 3A, the SC capacitive electrode 30 comprises a capacitive contact surface 32, electrode shield 34, amplifier 36, lead 38 and housing 31.

According to the invention, the capacitive contact surface 32 can comprise any conventional electrode surface that is adapted to receive capacitance potential signals from a subject when disposed proximate thereto.

In some embodiments, the capacitive contact surface 32 comprises a flexible electrode that is configured to conform to a subject's anatomy.

According to the invention, the amplifier 36 can comprise any suitable amplifier that is configured to amplify signals received by the SC capacitive electrode 30, e.g., capacitance potential signals, and transmit the amplified signals to control module 22 via lead 38.

In some embodiments, the amplifier 36 comprises an input impedance in the range of 10⁶Ω-10⁹Ω (1 MΩ-1 GΩ). In a preferred embodiment, the integrated amplifier 36 comprises an impedance in the range of 10⁶-10⁷Ω.

According to the invention, the lead 38 can comprise any suitable lead that is configured to provide a pathway for electrical signals, e.g., capacitance potential signals.

According to the invention, the housing 31 of the SC capacitive electrode 30 can comprise any convention housing that is configured to receive and contain a capacitive electrode without introducing interference.

In some embodiments, the SC capacitive electrode 30 comprises a flexible circuit incorporated therein that is in communication with the capacitive contact surface 32, integrated amplifier 36 and lead 38.

Referring now to FIG. 3B, there is shown another embodiment of a capacitive electrode comprising a multi-layer fabric electrode 40. As illustrated in FIG. 3B, the multi-layer fabric electrode 40 preferably comprises top and bottom conductive fabric layers 42a, 42b, a central polymer layer 46 having a thickness “t” and top and bottom intermediate fabric layers 44a, 44b.

In some embodiments, the top and bottom conductive fabric layers 42a, 42b preferably comprise a conductive fabric material. According to the invention, the conductive fabric material can comprise any fabric material, e.g., cotton, nylon, polyester and polyurethane based fabrics, which is coated and/or embedded with at least one electrically conductive element, e.g., carbon, nickel, gold, silver, titanium and combinations thereof.

According to the invention, suitable conductive fabric materials include, without limitation, silver coated polyesters, carbon-impregnated polymers and conductive polyparaphenylene terephthalamide polymer (ARACON®).

According to the invention, the central polymer layer 46 can comprise any insulating dielectric material, including, without limitation, polytetrafluoroethylene (PTFE), polyethylene (PE), polyimide, polypropylene, polystyrene and combinations thereof.

In some embodiments of the invention, the central polymer layer 46 comprises an absorbent dielectric polymer, e.g., sodium polyacrylate, polyacrylamide copolymer or a combination thereof, that is adapted to absorb any aqueous solution, e.g., water, on contact and, thus, maintain a constant relative humidity in the range of 55-100%, more preferably, in the range of 55-60% to reduce interference caused by to humidity-related static charge.

According to the invention, the central polymer layer 46 can also comprise any suitable thickness (denoted “t” in FIG. 3B). In some embodiments, the central polymer layer 46 comprises a thickness in the range of 0.1-10 mm, more preferably, the central polymer layer 46 comprises a thickness in the range of 0.5-2 mm.

In some embodiments, the top and bottom intermediate fabric layers 44a, 44b comprise one of the aforementioned fabric materials, such as cotton.

In some embodiments, the intermediate fabric layers 44a, 44b are included in the multi-layer fabric electrode 40 structure, as shown in FIG. 3B.

In some embodiments, the multi-layer fabric electrode 40 comprises a flexible circuit that is in communication with the top and bottom conductive fabric layers 42a, 42b.

Referring back to FIG. 2, as indicated above, in a preferred embodiment, the sensor sub-system 21 further comprises a conductive layer 5. In a preferred embodiment, the conductive layer 5 is sized and configured to be disposed proximate, more preferably, over, the first and second capacitive electrodes 2a, 2b.

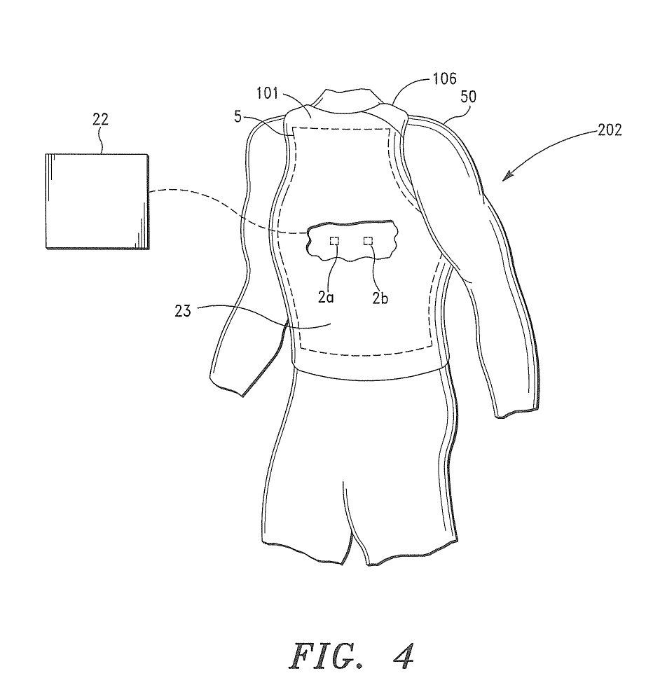

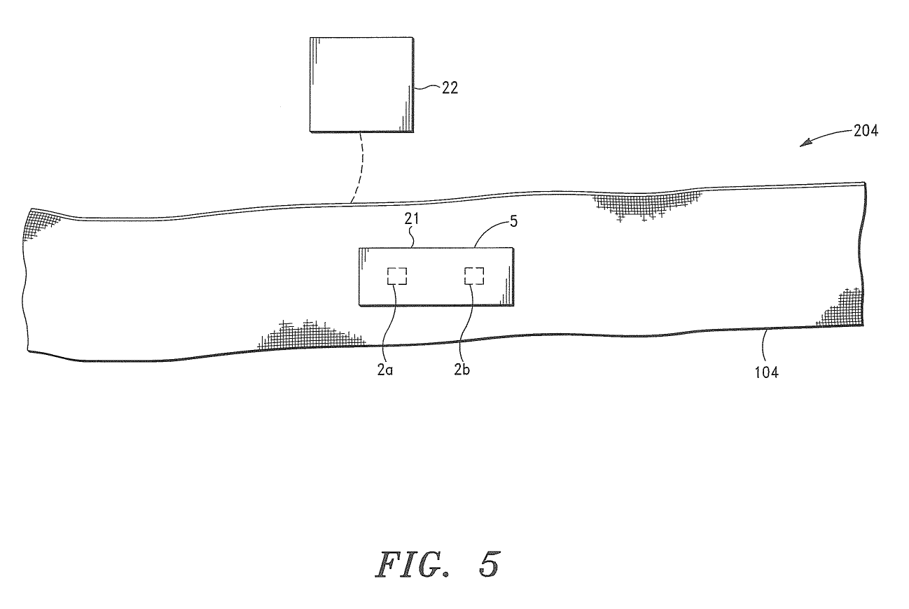

In some embodiments, the conductive layer 5 comprises a conductive material that is configured to be incorporated into or woven into a wearable physiological monitoring system, such as the embodiments of wearable physiological monitoring systems 202 and 204 shown in FIGS. 4 and 5, and described in detail in Applicant's Co-pending U.S. application Ser. Nos. 15/491,775 and 15/491,808, which are incorporated by reference herein. According to the invention, the conductive material can comprise, without limitation, any of the aforementioned conductive fabric materials.

According to the invention, the sensor sub-system 21 can comprise any number of conductive layers. In some embodiments, the sensor sub-system 21 comprises a plurality of conductive layers.

In a preferred embodiment, the sensor sub-system 21 comprises a single conductive layer.

In some embodiments, the conductive layer 5 comprises a flexible and/or stretchable material. In a preferred embodiment, the conductive layer 5 is configured to flex and/or stretch along at least two axes.

In some embodiments, the conductive layer 5 preferably comprises an area in the range of two (2) to five hundred (500) times the area of the first and second capacitive electrodes 2a, 2b, more preferably, the conductive layer 5 preferably comprises an area in the range of two (2) to twenty (20) times the area of the first and second capacitive electrodes 2a, 2b.

In a preferred embodiment of the invention, the conductive layer 5 is adapted and configured to (i) couple to an anatomical region of a subject, wherein the anatomical region forms a capacitive reference site, and (ii) when coupled to the capacitive reference site, generate at least one anatomical reference signal that reflects the electrical potential between the conductive layer 5 and anatomical structures disposed proximate the anatomical region (i.e. capacitive reference site).

According to the invention, the conductive layer 5 can be coupled to any anatomical region of a subject. The conductive layer 5 can also be coupled to a plurality of anatomical regions.

In a preferred embodiment of the invention, the conductive layer 5 is coupled to the torso, i.e. the thoracic and abdominal regions, of a subject.

In a preferred embodiment of the invention, the control module 22, i.e. processing system 8 thereof, is programmed and configured to receive the capacitance potential signals, capacitance potential noise signals, and anatomical reference signals provided by the sensor sub-system 21 (and sensor sub-system 23, discussed below).

In a preferred embodiment of the invention, the control module 22, i.e. processing system 8 thereof, is further programmed and configured to determine (i) capacitance potential parameters as a function of the capacitance potential signals, (ii) capacitance potential noise parameters as a function of the capacitance potential noise signals, (iii) anatomical reference parameters as a function of the anatomical reference signals, and (iv) at least one average anatomical reference parameter as a function of the anatomical reference parameters.

In a preferred embodiment of the invention, the control module 22, i.e. processing system 8 thereof, is further programmed and configured to determine (i) at least one capacitive reference parameter as a function of the capacitance potential parameters, at least one average anatomical reference parameter, and at least one capacitance potential noise parameter, (ii) at least one ECG pattern as a function of the capacitance potential parameters, capacitive reference parameter, and at least one of the capacitance potential noise parameters, and (iii) at least one physiological characteristic of the monitored subject as a function of the ECG pattern.

According to the invention, various physiological characteristics can be determined as a function of the ECG pattern, including, but not limited to, heart rate and various seminal cardiac cycle parameters, e.g., atrial systole contraction pulse, peak of ventricular contraction and recovery of the left and right ventricles.

In a preferred embodiment of the invention, the control module 22, i.e. processing system 8 thereof, is programmed and configured to determine a plurality of ECG patterns over time that represent the electro-physiologic depolarization and repolarization patterns of a subject's heart during at least one cardiac cycle.

In some embodiments, the control module 22, i.e. processing system 8 thereof, is further programmed and configured to transmit signals representing ECG patterns, i.e. ECG signals, to a remote receiving device that is configured to generate a visual representation of the ECG patterns over time, e.g., electro-physiologic depolarization and repolarization patterns of a subject's heart. The ECG patterns can then be assessed by a medical professional to determine any disease and/or physiological malignancy represented by the ECG patterns, e.g., ST elevated myocardial infarction (STEMI) of a subject's heart.

In a preferred embodiment, the conductive layer 5 is configured to provide an electromagnetic shield for at least one component of the physiological monitoring system 105, preferably, provide an electromagnetic shield for at least the first and second capacitive electrodes 2a, 2b.

According to the invention, the conductive layer 5 preferably reduces external electromagnetic interference received by the first and second capacitive electrodes 2a, 2b, such as electromagnetic interference due to the magnetic fields of power lines, by shielding the first and second capacitive electrodes 2a, 2b.

According to the invention, by positioning the first and second capacitive electrodes 2a, 2b between the conductive layer 5 and the skin of a monitored subject, signal interference due to the displacement of the electrode-to-skin distance is also reduced.

In some embodiments, the conductive layer 5 is further configured to physically shield at least one component of the physiological monitoring system 102; particularly, the first and second capacitive electrodes 2a, 2b, from humidity to reduce humidity-related static interference. Preferably, the conductive layer 5 is configured to effectively shield the first and second capacitive electrodes 2a, 2b in low relative humidity environments having a relative humidity less than 50%.

It is well established that, when a fabric or garment is in direct contact with a subject in a low relative humidity environment, the fabric or garment will often generate static electricity via the triboelectric effect.

According to the invention, the conductive layer 5 can thus be further configured to maintain an internal environment between the conductive layer 5 and the surface of a subject at a relative humidity of at least 50% or greater to reduce humidity-related static interference.

Referring again to FIG. 2, in a preferred embodiment, the electrical activity monitoring system 105 further includes a control module 22. As indicated above, the control module 22 preferably comprises processing system 8, which is programmed and configured to control the sensor sub-system 21 and the function thereof, and the transmission and receipt of signals therefrom.

As indicated above, in preferred embodiment of the invention, the processing system 8 and, hence, control module 22 is further programmed and configured to determine (i) capacitance potential parameters as a function of the capacitance potential signals, (ii) capacitance potential noise parameters as a function of the capacitance potential noise signals, (iii) anatomical reference parameters as a function of the anatomical reference signals, (iv) at least one average anatomical reference parameter as a function of the anatomical reference parameters, and determine physiological information associated with a monitored subject as a function of the signals, including at least one physiological parameter or characteristic, e.g. heart rate.

In some embodiments of the invention, the processing system 8 also includes a “rules set” that includes a rule in which an alert signal is generated and transmitted if a signal or signals from the sensor sub-system 21 or generated by the processing system 8 indicate that a physiological parameter or characteristic of a monitored subject is outside a predetermined range.

In a preferred embodiment, the processing system 8 further comprises at least one signal interference and/or noise reducing algorithm, including, without limitation wavelet neural network algorithms, adaptive filtering algorithms, least mean squared algorithms, normalized least mean squared algorithms, sign error least mean squared algorithms, sign data least mean squared algorithms, sign least mean squared algorithms and recursive least mean squared algorithms.

In some embodiments, the control module 22 further comprises a signal filtering means, such as a bandpass filter.

Referring back to FIG. 2, in some embodiments, the electronic module 22 preferably further comprises an amplifier system 9 that is designed and configured to amplify signals generated (or. provided) by the sensor sub-system 21, i.e. capacitance potential and/or capacitance reference signals.

In some embodiments, the amplifier system 9 comprises an input impedance in the range of 10⁶Ω-10⁹Ω (1 MΩ-1 GΩ).

In some embodiments, the amplifier system 9 comprises an integrated amplifier that is incorporated into a capacitive electrode, such as amplifier 36 of self-contained electrode 30 shown in FIG. 3A.

In a preferred embodiment of the invention, the control module 22 further comprises a data transmission system 14. Preferably, the data transmission system 14 comprises a transmitter that is programmed and configured to wirelessly transmit processed signals representing electrical activity of a subject that is generated by the control module 22 to a remote signal receiving device (not shown), e.g., a base module or a hand-held electronic device, such as a smart phone, tablet, computer, wearable electronics device, etc.

As illustrated in FIG. 2, in a preferred embodiment, the electrical activity monitoring system 105 further comprises display means 12 and is programmed and configured to display received and/or processed signals and/or generated ECG patterns by the control module 22. In some embodiments, the display means 12 comprises a display screen that is configured to display a user interface that provides a visual representation of processed signals and generated ECG patterns representing electrical activity and, thereby, physiological information associated with a monitored subject.

According to the invention, the control module 22 can also be configured to communication with an external interface or program, such as a website accessible over a network, to display and store the electrical activity data and patterns.

In some embodiments of the invention, the electrical activity monitoring system 105 is further configured to monitor one or more additional physiological characteristics associated with a monitored subject. Thus, in some embodiments, the electrical activity monitoring system 105 further includes one or more additional physiological sensors, such as a pulse oximeter (Sₚ0₂) or core body temperature sensor (shown in phantom and denoted “13” in FIG. 2), which are in communication with the signal transmission conductors 6.

Referring now to FIGS. 4 and 5, two (2) embodiments of electrical activity monitoring systems of the invention will be described in detail.

Referring first to FIG. 4, there is shown one embodiment of a wearable electrical activity monitoring system 202. As illustrated in FIG. 4, the wearable electrical activity monitoring system 202 comprises wearable garment 101, another embodiment of a sensor sub-system 23 and control module 22.

As also illustrated in FIG. 4, in the noted embodiment, the wearable garment 101 is preferably configured to cover at least the chest region and upper back of a subject 50. According to the invention, the wearable garment 101 can, however, also be configured to cover other regions of the subject 50, including, without limitation, the lower abdominal region.

According to the invention, the wearable garment 101 can comprise any suitable fabric, such as cotton, polyester and/or polyurethane based fabrics.

In some embodiments of the invention, at least one of the shoulder portions 106 of the wearable garment 101 comprises a two-piece portion, i.e. an over-lapping strap configuration, to facilitate easy placement of the garment 101 on a subject, e.g., elderly subject. In the noted embodiments, the two-piece portion includes a conventional Velcro® system or hooks or snaps to secure the ends of the over-lapping strap after the garment 101 is positioned on the subject's body.

As further illustrated in FIG. 4, the sensor sub-system 23 comprises a conductive layer 5 that is incorporated into wearable garment 101 and disposed over capacitive electrodes 2a, 2b. In a preferred embodiment, the capacitive electrodes 2a, 2b each comprise a self-contained electrode with an integrated amplifier, such as self-contained electrode 30 shown in FIG. 3A.

In some embodiments, the capacitive electrodes 2a, 2b each comprise a fabric electrode, such as fabric electrode 40 shown in FIG. 3B.

In some embodiments, the wearable garment 101 further comprises integral signal transmission conductors 6 that are in communication with the sensor sub-system 23 and control module 22, including components thereof. In a preferred embodiment, the signal transmission conductors 6 are disposed in a flexible configuration thereon.

In some embodiments, the signal transmission conductors 6 comprise a conventional insulated conductive wire, such as a copper wire insulated by a rubber coating.

In some embodiments of the invention, the signal transmission conductors 6 comprise one of the aforementioned conductive fabrics.

In some embodiments, the signal transmission conductors 6 comprise a thin linear member, e.g., thread or chord, which is wrapped with a conductive wire. Preferably, the linear member comprises a stretchable member, i.e. is at least partially constructed of a stretchable material, and the wire is spirally wrapped around the stretchable member.

In a preferred embodiment, the signal transmission conductors 6 are adapted to receive signals from the sensor sub-system 23, i.e. capacitance potential signals, capacitance potential noise signals and anatomical reference signals, and transmit the capacitance potential signals, capacitance potential noise signals and anatomical reference signals to the control module 22.

In some embodiments, the control module 22 is incorporated into and/or disposed on the wearable garment 101.

As discussed in detail above, in a preferred embodiment, the control module 22 comprises processing system 8 that is programmed and configured, i.e. includes programs, instructions and associated algorithms and parameters, to control the sensor sub-system 23 and, hence, the first and second capacitive electrodes 2a, 2b, conductive layer 5 and the functions thereof, and the transmission and receipt of signals therefrom, as well as the data transmission system 14.

As also discussed in detail above, the processing system 8 is also preferably programmed and configured to process signals generated (and/or provided) and transmitted by the sensor sub-system 23, and determine physiological information associated with the monitored subject as a function of the signals.

Referring now to FIG. 5, there is shown another embodiment of a wearable electrical activity monitoring system 204. As illustrated in FIG. 5, the wearable electrical activity monitoring system 204 includes wearable band 104, sensor sub-system 21 and control module 22.

As indicated above, in some embodiments, the garment band 104 illustrated in FIG. 5 and discussed in detail above is configured to be positioned on a subject, i.e. wrap around a subject. According to the invention, the wearable garment band 104 can comprise any suitable fabric, including one of the aforementioned fabrics, such as cotton, nylon, polyester and polyurethane based fabrics.

In some embodiments, the wearable garment band 104 further comprises integral signal transmission conductors 6 that are in communication with the sensor sub-system 21 and control module 22, including components thereof. In a preferred embodiment, the signal transmission conductors 6 are disposed in a flexible configuration thereon.

In some embodiments, the control module 22 is incorporated into and/or disposed on the wearable garment band 104.

As indicated above, according to the invention, the electrical activity monitoring systems of the invention can also be employed in other apparatuses and/or systems, such the apparatuses and systems disclosed in Applicant's U.S. application Ser. Nos. 13/854,280, 15/491,775 and 15/491,808, which are expressly incorporated by reference herein in their entirety.

According to the invention, the electrical activity monitoring systems of the invention can thus be readily employed to monitor electrical activity of a subject and determine physiological characteristics of the subject as a function of the subject's electrical activity.

In some embodiments, the method for determining a physiological characteristic of a subject comprises:
(i) providing an electrical activity monitoring system of the invention, the system comprising first and second capacitive electrodes, a conductive layer and a control module;
(ii) positioning the electrical activity monitoring system on a subject's body, wherein the first and said second capacitive electrodes are electrically coupled to the subject's body, and the conductive layer is coupled to an anatomical region of the subject;
(iii) initiating the electrical activity monitoring system;
(iv) detecting first and second capacitance potential signals with the first and second capacitive electrodes;
(v) detecting capacitance potential noise signals with the first and second capacitive electrodes;
(vi) generating anatomical reference signals with the conductive layer;
(vii) transmitting the first and second capacitance potential signals, capacitance potential noise signals, and anatomical reference signals to the control module;
(viii) determining a first capacitive potential parameter as a function of the first capacitive potential signal;
(ix) determining a second capacitive potential parameter as a function of the second capacitive potential signal;
(x) determining capacitive potential noise parameters as a function of the capacitive potential noise signals;
(xi) determining anatomical reference parameters as a function of the anatomical reference signals;
(xi) determining an average anatomical reference parameter as a function of the anatomical reference parameters;
(xii) determining a capacitive reference parameter as a function of the first and second capacitance potential parameters, average anatomical reference parameter, and at least one of the capacitance potential noise parameters;
(x) determining at least one ECG pattern as a function of the first and second capacitance potential parameters, capacitive reference parameter, and at least one of the capacitance potential noise parameters; and
(xi) determining at least one physiological characteristic associated with the monitored subject as a function of at least one determined ECG pattern.

By virtue of the unique sensor sub-system, and conductive layer thereof, and capacitive reference site provided thereby, and unique positioning of the capacitive electrodes between the conductive layer and the monitored subject, the wearable electrical activity monitoring systems described above provide several unexpected superior results and advantages. Among the advantages are the following:
The provision of wearable electrical activity monitoring systems that detect signals representing electrical activity of a subject with enhanced accuracy and record signals representing same that exhibit enhanced signal quality.
The provision of wearable electrical activity monitoring systems that provide substantially larger capacitive reference site(s) than conventional capacitive reference sites employed with conventional physiological monitoring systems. By virtue of the larger capacitive reference site(s), the wearable electrical activity monitoring systems substantially reduce signal baseline wandering, low-frequency artifacts and signal noise.
The provision of wearable electrical activity monitoring systems and methods that substantially reduce or abate external electromagnetic interference, e.g., electromagnetic radiation from any powered device, power lines and interstellar sources, and humidity-induced interference.
The provision of wearable electrical activity monitoring systems that substantially reduce or abate internal interference, e.g., interference due to changes in displacement of the electrode-to-skin distance and friction between the electrodes and the medium that the electrodes are attached to.
The provision of wearable electrical activity monitoring systems that provide a controlled internal environment that reduces or abates humidity-related static interference.
The provision of improved methods for accurately determining one or more physiological characteristics associated with a subject as a function of detected electrical activity.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An electrical activity monitoring system, comprising:
a sensor sub-system that is configured to be positioned proximate to a subject's body, wherein said sensor sub-system is in communication with said subject's body,
said sensor sub-system comprising at least first and second capacitive electrodes and a conductive layer, said conductive layer being disposed adjacent said first and second capacitive electrodes,
said first capacitive electrode being adapted to provide at least a first capacitance potential signal representing a first capacitance potential between said first capacitive electrode and said subject's body, when said first capacitive electrode is electrically coupled to said subject's body,
said second capacitive electrode being adapted to provide at least a second capacitance potential signal representing a second capacitance potential between said second capacitive electrode and said subject's body, when said second capacitive electrode is electrically coupled to said subject's body,
said first and second capacitive electrodes being further adapted to detect and receive capacitance potential noise signals emanating from said subject's body, when said first and second capacitive electrodes are electrically coupled to said subject's body,
said conductive layer being adapted to couple to an anatomical region of said subject, wherein said anatomical region forms a capacitive reference site, and wherein said conductive layer generates anatomical reference signals, when said conductive layer is electrically coupled to said subject's body, said anatomical reference signals representing electrical potentials between said conductive layer and anatomical structures proximate said capacitive reference site,
said conductive layer being further adapted to shield said first and second capacitive electrodes from electromagnetic interference and humidity-related static interference, when said ECG system is in said communication with said subject's body;
a signal transmission conductor, said signal transmission conductor being in communication with said first and second capacitive electrodes, said conductive layer and said control module,
said signal transmission conductor being adapted to receive said at least first and second capacitance potential signals, anatomical reference signals, and capacitance potential noise signals,
said signal transmission conductor being further adapted to transmit said at least first and second capacitance potential signals, said anatomical reference signals, and said capacitance potential noise signals to said control module; and
a control module, said control module being programmed and configured to receive said at least first and second capacitance potential signals, said anatomical reference signals, and said capacitance potential noise signals,
said control module being further programmed and configured to determine a first capacitance potential parameter as a function of said first capacitance potential signal, a second capacitance potential parameter as a function of said second capacitance potential signal, capacitance potential noise parameters as a function of said capacitance potential noise signals, anatomical reference parameters as a function of said anatomical reference signals, and at least one average anatomical reference parameter as a function of said anatomical reference parameters,
said control module being further programmed and configured to determine at least one capacitive reference parameter as a function of said first and second capacitance potential parameters, said at least one average anatomical reference parameter, and said capacitance potential noise parameters,
said control module being further programmed and configured to generate at least one ECG pattern as a function of said first and second capacitance potential parameters, said at least one capacitive reference parameter, and said capacitance potential noise parameters, and determine at least one physiological characteristic of said subject as a function of said at least one ECG pattern.

2. The electrical activity monitoring system of claim 1, wherein said conductive layer comprises a conductive fabric material.

3. The electrical activity monitoring system of claim 1, wherein said at least one anatomical region comprises said subject's torso.

4. The electrical activity monitoring system of claim 1, wherein said control module further comprises an amplifier system that is adapted to amplify said first and second capacitance potential signals.

5. The electrical activity monitoring system of claim 1, wherein said system further comprises at least one additional physiological sensor that is adapted to monitor a physiological parameter of said subject.

6. A method of determining a physiological characteristic of a subject, comprising the steps of:
(i) providing an electrical activity monitoring system, said electrical activity monitoring system comprising a sensor sub-system that is configured to be positioned proximate to a subject's body, wherein said sensor sub-system is in communication with said subject's body, said sensor sub-system comprising at least first and second capacitive electrodes and a conductive layer, said conductive layer being disposed adjacent said first and second capacitive electrodes,
said first capacitive electrode being adapted to provide at least a first capacitance potential signal representing a first capacitance potential between said first capacitive electrode and said subject's body, when said first capacitive electrode is electrically coupled to said subject's body,
said second capacitive electrode being adapted to provide at least a second capacitance potential signal representing a second capacitance potential between said second capacitive electrode and said subject's body, when said second capacitive electrode is electrically coupled to said subject's body,
said first and second capacitive electrodes being further adapted to detect and receive capacitance potential noise signals emanating from said subject's body, when said first and second capacitive electrodes are electrically coupled to said subject's body,
said conductive layer being adapted to couple to an anatomical region of said subject, wherein said anatomical region forms a capacitive reference site, and wherein said conductive layer generates anatomical reference signals, when said conductive layer is electrically coupled to said subject's body, said anatomical reference signals representing electrical potentials between said conductive layer and anatomical structures proximate said capacitive reference site,
said conductive layer being further adapted to shield said first and second capacitive electrodes from electromagnetic interference and humidity-related static interference, when said ECG system is in said communication with said subject's body,
a signal transmission conductor, said signal transmission conductor being in communication with said first and second capacitive electrodes, said conductive layer and said control module,
said signal transmission conductor being adapted to receive said at least first and second capacitance potential signals, anatomical reference signals, and capacitance potential noise signals,
said signal transmission conductor being further adapted to transmit said at least first and second capacitance potential signals, said anatomical reference signals, and said capacitance potential noise signals to said control module, and
a control module, said control module being programmed and configured to receive said at least first and second capacitance potential signals, said anatomical reference signals, and said capacitance potential noise signals,
said control module being further programmed and configured to determine a first capacitance potential parameter as a function of said first capacitance potential signal, a second capacitance potential parameter as a function of said second capacitance potential signal, capacitance potential noise parameters as a function of said capacitance potential noise signals, anatomical reference parameters as a function of said anatomical reference signals, and at least one average anatomical reference parameter as a function of said anatomical reference parameters,
said control module being further programmed and configured to determine at least one capacitive reference parameter as a function of said first and second capacitance potential parameters, said at least one average anatomical reference parameter, and said capacitance potential noise parameters,
said control module being further programmed and configured to generate at least one ECG pattern as a function of said first and second capacitance potential parameters, said at least one capacitive reference parameter, and said capacitance potential noise parameters, and determine at least one physiological characteristic of said subject as a function of said at least one ECG pattern;
(ii) positioning said electrical activity monitoring system on a subject's body, wherein said sensor sub-system is in communication with said subject's body, said first and said second capacitive electrodes are electrically coupled to said subject's body, and said conductive layer being is coupled to an anatomical region of said subject;
(iii) initiating said electrical activity monitoring system;
(iv) detecting third and fourth capacitance potential signals with said first and second capacitive electrodes;
(v) detecting first capacitance potential noise signals with said first and second capacitive electrodes;
(vi) generating first anatomical reference signals with said conductive layer;
(vii) transmitting said third and fourth capacitance potential signals, said first capacitance potential noise signals, and said first anatomical reference signals to said control module;
(viii) determining a third capacitive potential parameter as a function of said third capacitive potential signal;
(ix) determining a fourth capacitive potential parameter as a function of said fourth capacitive potential signal;
(x) determining first capacitive potential noise parameters as a function of said first capacitive potential noise signals;
(xi) determining first anatomical reference parameters as a function of said first anatomical reference signals;
(xii) determining an average anatomical reference parameter as a function of said anatomical reference parameters;
(xiii) determining a capacitive reference parameter as a function of said first and second capacitance potential parameters, said average anatomical reference parameter, and at least one of said first capacitance potential noise parameters;
(xiv) determining at least one ECG pattern as a function of said first and second capacitance potential parameters, said capacitive reference parameter, and said at least one of the capacitance potential noise parameters; and
(xv) determining at least one physiological characteristic associated with said subject as a function of said at least one ECG pattern.

7. The method of claim 6, wherein said first physiological characteristic comprises atrial systole contraction pulse.

8. The method of claim 6, wherein said first physiological characteristic comprises peak ventricular contraction.
